(19) United States
(12) Patent Application Publication
(10) Pub. No.: US 2019/0111219 A1
(43) Pub. Date: Apr. 18, 2019

(54) Multi-chamber Medicament Vaporizer Device

(71) Applicant: Eric Franquez Campos, Fullerton, CA (US)

(72) Inventor: Eric Franquez Campos, Fullerton, CA (US)

(21) Appl. No.: 16/160,956

(22) Filed: Oct. 15, 2018

Related U.S. Application Data
(60) Provisional application No. 62/707,002, filed on Oct. 16, 2017.

(51) Int. Cl. A61M 11/04; A61K 36/185

(52) U.S. Cl. CPC A61M 11/041; A61K 36/185; A61M 2205/3576; A61M 2205/584; A61M 2205/8206; A61M 2205/587; A61M 2205/505

(57) ABSTRACT

A hand held Multi-chamber medicament vaporizer device comprising a body configured for accepting multiple medicament cartridges. A microprocessor controlled circuit may serve as a controller for the device.

### TECHNICAL FIELD

This invention relates to a vaporizer device and method of delivering two or more medicaments or agents from isolated reservoirs using a vaporizer device with two or more separate chambers or reservoirs. More specifically, the present invention is directed to a dual or multi-chamber module comprising a separate reservoir housing a medicament and at least a second separate reservoir housing another medicament. The separate chambers may provide a user an option of micro-dosing each medicament to a custom level dictated by the user's tolerance or health care professional. The medicaments may be available in two or more chambers each containing independent (single molecule compounds) or pre-mixed (co-formulated multiple molecule compounds). Additionally, the present invention may be optimized for a specific target patient group through control of a dosing and definition of a therapeutic profile.

### BACKGROUND

Cannabis is a substance derived from the hemp plant (Cannabis indica or Cannabis sativa). Derived materials include cannabinoids, terpenes and flavonoids. In this specification these, and other derivatives, are referred to generically as cannabis molecules.

After decarboxylation, Cannabidiol (CBD) and tetrahydrocannabinol (THC) are the two main ingredients in the cannabis plant. Both CBD and THC belong to a unique class of compounds known as cannabinoids.

THC is probably best known for being the psychoactive ingredient in cannabis. CBD, however, is non-psychoactive. In other words, CBD can't get you high. While disappointing to recreational users, this unique feature of CBD is what makes it appealing as a medicine.

Certain mood disorders require treatment using one or more different medicaments. The sensitivity to drugs for people with mood disorders is common and there is no “one size fits all” solution to the problem. Cannabinoid treatment for mood disorders is a relatively new science, however, it is known that too much cannabis can negatively affect the condition and patient being treated.

Patients prefer and/or require cannabinoid and terpene compounds to be delivered in a specific relationship with each other in order to deliver the optimum therapeutic dose. This invention is of particular benefit where combination therapy is desirable, but not possible in a single formulation for reasons such as, but not limited to, stability, dose amount, dose control and compromised therapeutic performance and toxicology.

Micro-dosing cannabis involves taking a very measured micro-dose of THC, the psychoactive ingredient, and a measured micro-dose of CBD, the non-psychoactive therapeutic ingredient in cannabis.

With micro-dosing, users get the maximum benefit from the minimum amount, without becoming overly affected.

Currently, anyone that wants to micro-dose faces two hurdles: finding the right minimum dose and finding products that will deliver it.

The ability to set a dose of one cannabinoid (such as THC) to a custom level and also having ability to custom set the dose of a separate cannabinoid or mixture of cannabinoids (such as CBD) may give the healthcare professionals and patients the flexibility to titrate the medicinal compounds up or down with more precision. The present invention may give the opportunity for precisely varying the quantity of one or both reservoirs of cannabinoids. For example, one fluid quantity may be varied by changing the programming of the vaporizer device. This may be completed by dialing a user variable dose or changing the device's “fixed” dose. The second medicament quantity may be changed exactly the same way. The user or healthcare professional may then select the most appropriate micro-dosing quantity for a particular treatment regime.

The main problem with cannabis based vaporizer pen oil cartridges are the predetermined THC/CBD ratios supplied by the manufacturers. The end user has no control over how much THC and or other cannabinoids they consume through using the single chambered vaporizer pens. Cannabinoids (CBD, THC, etc.) are hydrophobic (water-hating) oily substances and, as such, are not water-soluble. They can, however, be formulated to be water-compatible and appear water soluble. The term “water-soluble CBD” has lately been used extensively in the medical cannabis industry. Today, CBD, THC and other oils can be made water-compatible if they are formulated as oil-in-water nano-emulsions, which are stable and visually homogeneous oil/water mixtures. Nanoemulsions can be prepared in concentrated forms (tens of mg/ml) that are fully miscible with water and, therefore, appear water-soluble. In addition, nanoemulsions can be made translucent and practically tasteless, which means that they can be mixed into water without compromising its optical clarity or taste. The benefits of nanoemulsions include: easily mixed into beverages, translucent, low-taste and safe for consumption, exceptionally high bioavailability, fast onset of action and stable blood level and high CBD/THC loading capacity.

These and other advantages will become evident from the following more detailed description of the invention.

The present invention combines the Multi-chamber vaporizer device to provide a precise, high water soluble and bioavailable product for medicinal cannabis consumption.

These and other advantages will become evident from the following more detailed description of the invention.

### SUMMARY OF THE INVENTION

The present application may disclose a device that allows for the dose controlled multi-chamber vaporizer of medicaments within a single vaporizer device. Preferably, such a vaporizer device may include two or more separate oil chambers individually attached to a single mouthpiece. Each chamber may be attached to a software driven temperature and timing controller used for precise vaporizer of liquid compounds. A user may set and vaporizer a predetermined dose of compounds through one chamber and set a different dose to vaporizer from the other chamber. Preferably, the multi-chamber vaporizer interface may be programmed to vaporize each chamber with the preset microdose. This multi-chambered dose interface may control the voltage, temperature and timing of the heating coils of the device such that a predefined quantity of cannabinoid compounds may be vaporized when each chambered dose is set and vaporized through the mouthpiece. By defining the therapeutic relationship between the individual cannabinoid compounds this vaporizer device would help ensure that a user receives the optimum therapeutic combination dose from a multi-cannabinoid compound vaporizer device without the inherent risks associated with single dose vaporizer pen devices. The cannabinoids and terpenes can be water soluble fluids that make it easy to heat up and vaporize in a shorter amount of time.

### BRIEF DESCRIPTION OF THE DRAWINGS

FIG. 1 shows a complete assembled multi-chamber medicament dispensing device 101.

FIG. 2 shows the front view of the Multi-chamber medicament vaporizer device with some internals shown.

FIG. 3 shows a side view of the Multi-chamber medicament vaporizer device.

FIG. 4 shows a cutaway view of the Multi-chamber medicament vaporizer device exposing the medicament chambers.

### DETAILED DESCRIPTION

In the Summary above and in this Detailed Description, and the claims below, and in the accompanying drawings, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used—to the extent possible—in combination with and/or in the context of other particular aspects and embodiments of the invention, and in the invention generally

The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also contain one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range including that number (which may be a range having an upper limit or no upper limit, depending on the variable being defined). For example, “at least 1” means 1 or more than 1. The term “at most” followed by a number is used herein to denote the end of a range, including that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose limits include both numbers. For example, “25 to 100” means a range whose lower limit is 25 and upper limit is 100, and includes both 25 and 100.

The present invention is directed to a vaporizer device for administering a precise, customized dose of two or more medicaments. Setting the dose of the medicaments by the user gives the patient more reliable control over the therapeutic outcome of micro-dosed therapy. These combined variable doses are preferable over single dose therapy. The device may include touch pad interface for simple UP and DOWN control of dosing device. The vaporizer device comprises at least two heater cores in each chamber, a dual mouthpiece at the end of each chamber, a printed circuit board assembly, a USB charging port, a battery pack, a front and rear housing, two cartridge/vial holders, a vapor dispense chamber, an end cap, indicator screens, LED indicator lights, adjustment UP and DOWN keypad buttons, a vaporizer activation “GO” button and an ON/OFF button. The front and rear housings cover the PCB assembly and the rechargeable battery but leave the cartridge holders exposed for simple cartridge replacement. The end cap covers the mouth piece after each use.

Externally the ON/OFF button controls the power of device which may time out after 30 seconds of without use. The indicator screens show the amount of the dose chosen by the UP and DOWN keypad buttons. The vaporizer activation button may activate the heater core process which precisely heats up each of the chambers of the medicaments. LED indicator lights may show the user when the liquid doses have been delivered by turning from green to red in color. In order to install the medicament chambers into the vaporizer device, the glass or plastic cartridges may be inserted into connecting assembly and locked in with locking mechanism. Each cartridge may be connected to a multi-spout mouthpiece for easy vaporizing. Internally, the cartridges show a heater core that will heat up the liquid content of that chamber. An end cap is shown that may cover the mouthpiece when not in use.

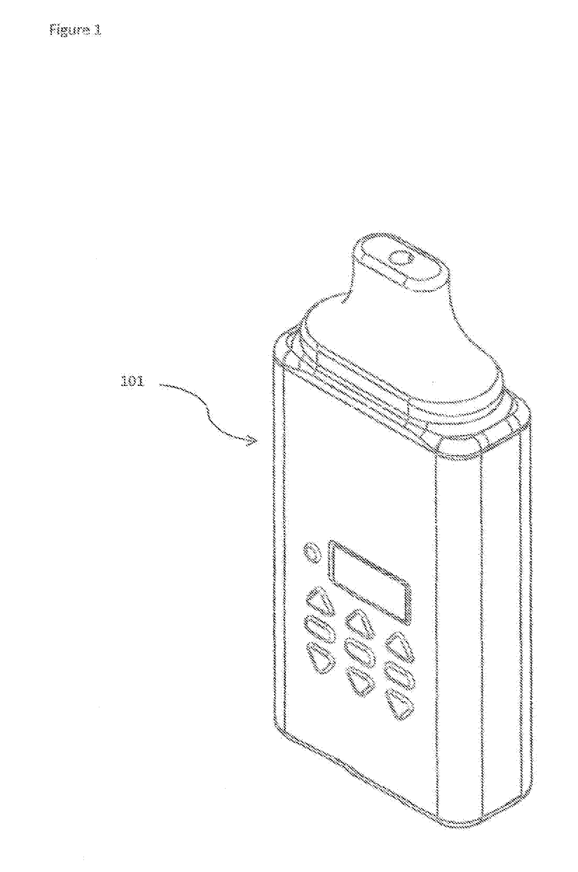

FIG. 1 shows a complete assembled multi-chamber medicament dispensing device 101.

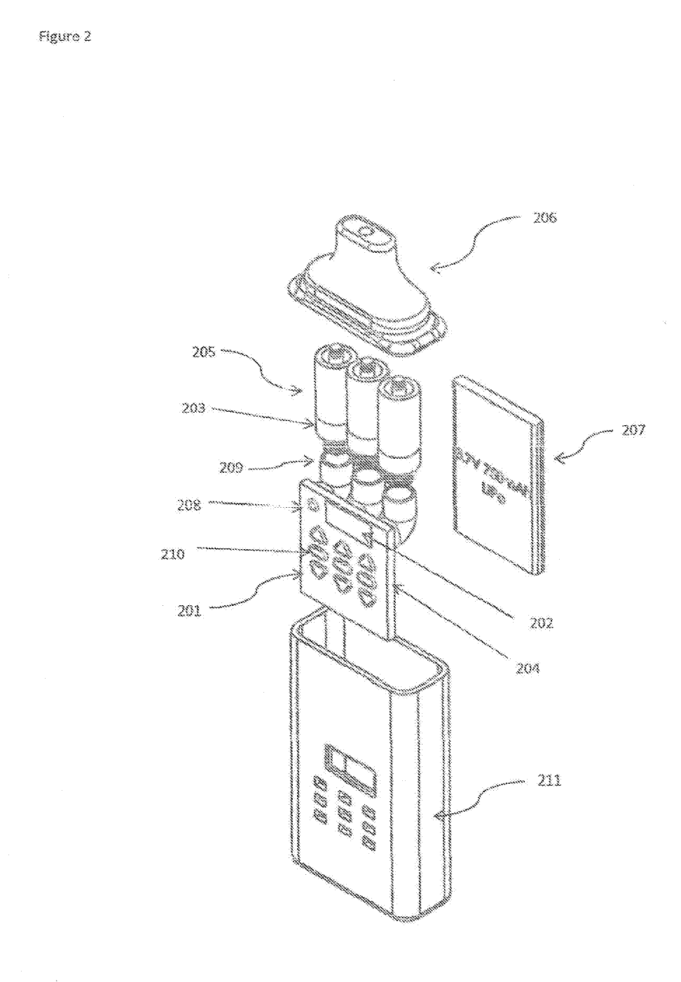

FIG. 2 shows the front view of the Multi-chamber medicament vaporizer device. UP and DOWN dose control buttons 201 are shown with a numbered indicator display 202. Vaporizing the medicament may be achieved by heating up the heater core 203 with a predetermined voltage and time sequence. The dose control buttons 201 may be operably connected to printed circuit board assembly 204 that engages the heater cores 203 of each cartridge 205 of the medicaments. A mouthpiece 206 is pictured at the top where the user may inflate the vaporized medicaments. A battery pack 207 may power the entire device. An On/Off button 208 controls the power. The heater core 203 fits onto a cartridge socket 209. LED indicator lights 210 may display the status of the device which may be contained in a housing 211.

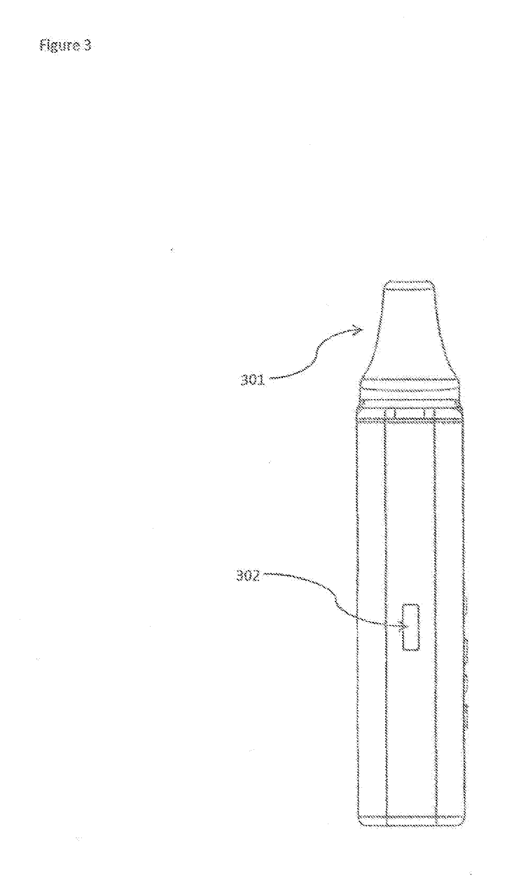

FIG. 3 shows a side view of the Multi-chamber medicament vaporizer device. The mouthpiece 301 is shown at the top and a charging port 302 is shown on the side.

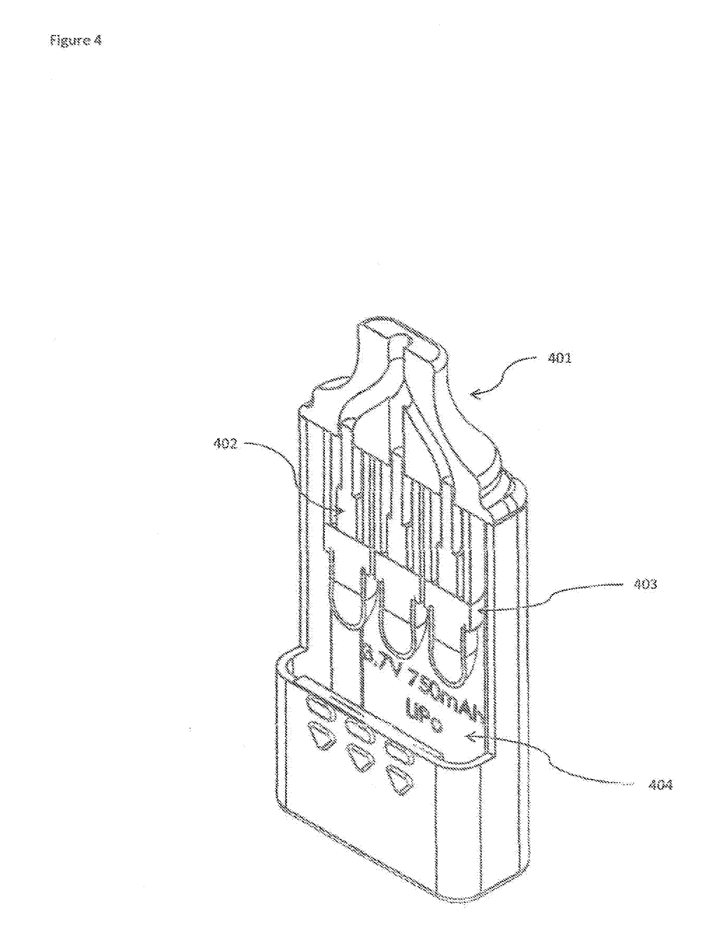

FIG. 4 shows a cutaway view of the Multi-chamber medicament vaporizer device. The mouthpiece 401 is shown fixed to the medicament cartridges 402. The heater cores 403 sit below the medicament cartridges 402. A battery pack 404 is shown behind the medicament cartridges 402 and heater cores 403.

This invention may be of particular benefit to patients with THC sensitivity and/or those who are not sure of what dose will work best for their condition. Giving the user the ability to titrate up or down in microdoses insures better patient tolerance and therapeutic benefits. This invention may eliminate the need for trial and error single dose consumption that current single chambered pen based systems offer.

In a preferred embodiment, the primary vaporizer device may have replaceable modules or cartridges and may be used more than once. It is possible to have a suite of different medicament modules for various conditions that may be prescribed. The vaporizer device may comprise a software driven, battery-powered printed circuit board coupled to two or more heating coils allowing for the software control of each precise dose. The invention is not limited to specific embodiments but comprises any combination of elements of different embodiments.

As mentioned above, in any of the methods described herein, appropriate oil compounds to be vaporized may be used. In general, the nanoemulsified lipid compounds may be cannabinoid oils or terpenes. The lipid compounds may comprise any active ingredient(s). For example, the lipid compound may comprise a botanical. The lipid compound may comprise a cannabinoid. The lipid compound may comprise one or more of: cetirizine, ibuprofen, naproxen, omeprazole, doxylamine, diphenhydramine, melatonin, or meclizine. The lipid compound may comprise one or more of: a polyphonel, short chain fatty acids, medium chain fatty acids, long chain fatty acids, a green tea catechin, caffeine, a phenol, a glycoside, a labdane diterpenoid, yohimbine, a proanthocyanidin, terpene glycoside, an omega fatty acid, echinacoside, an alkaloid, isovaleric acid, a terpene, gamma-aminobutyric acid, a senna glycoside, cinnamaldehyde, or Vitamin D.

Although the preceding description contains significant detail in relation to the preferred embodiment, it should not be construed as limiting the scope of the invention but rather as providing illustrations of the preferred embodiments.

Related applications include patent applications 20170231280A1, 20180000163A1, and 20170354180A1. These references do not teach the entire present invention.

The invention claimed is:

1. A Multi-chamber medicament vaporizer device comprising two or more medicament cartridges a system to vaporize each medicament.

2. The device of claim 1, controlled electronically.

3. The device of claim 1, wherein said cartridges may be interchanged and replaced with various medicaments.

4. The device of claim 1, wherein the cartridges provide a precise volume of medicaments.

5. The device of claim 1, wherein the cartridges provide a precise volume of cannabis-derived medicaments.

6. The device of claim 1, wherein the system to dispense each medicament is electronically tracked and communicated to another device.
